(19) United States
(12) Patent Application Publication
(10) Pub. No.: US 2015/0148726 A1
(43) Pub. Date: May 28, 2015

(54) Knee Brace Assembly

(71) Applicant: WB Development Company, LLC, Marlborough, MA (US)

(72) Inventor: Walter T. Bonin, Marlborough, MA (US)

(21) Appl. No.: 14/550,459

(22) Filed: Nov. 21, 2014

Related U.S. Application Data
(60) Provisional application No. 61/906,959, filed on Nov. 21, 2013.

(51) Int. Cl. A61F 5/01

(52) U.S. Cl. CPC A61F 5/0123
USPC 602/16

(57) ABSTRACT

A knee brace assembly includes a first brace assembly configured to be secured to a first leg portion of a leg relative to a knee and a second brace assembly configured to be secured to a second leg portion of the leg relative to the knee, the second leg portion opposing the first leg portion. The knee brace assembly includes a hinge disposed between the first brace assembly and the second brace assembly, the hinge having a plurality of strap elements each strap element defining a planar surface. The planar surfaces of the plurality of strap elements are disposed substantially parallel to each other along an anterior-posterior orientation of the first leg portion and the second leg portion.

### RELATED APPLICATIONS

This patent application claims the benefit of U.S. Provisional Application No. 61/906,959, filed on Nov. 21, 2013, entitled, “Knee Brace Assembly,” the contents and teachings of which are hereby incorporated by reference in their entirety.

### BACKGROUND

Knee braces are typically used by athletes engaged in vigorous physical activity to protect the knee from injury or to avoid worsening of an existing injury. Certain types of knee braces, termed prophylactic braces, are configured to minimize the initiation of an injury in an athlete. For example, prophylactic braces typically include a hinge joint disposed between upper and lower leg mounting supports where the hinge joint is aligned with a center of rotation of a user's knee joint. Generally, the prophylactic braces minimize excessive movement in the knee joint, either front to back (anterior-posterior), side to side (medial-lateral), or rotational, and aid in controlling knee stability.

### SUMMARY

Conventional knee braces suffer from a variety of deficiencies. For example, prophylactic braces include one or more hinge joints that allow a wearer to flex his leg at the knee joint along a substantially anterior-posterior direction. However, conventional hinge joints can inhibit medial-lateral movement of the wearer's leg which can constrain the natural positioning of the wearer's upper and lower leg portions during use. Additionally, in certain knee braces, the hinge joint is configured as disk hinge having a center of rotation substantially aligned with the center of rotation of the wearer's knee. However, the physiological motion of a wearer's knee includes both rotation and linear translation of the upper and lower leg portions relative to each other. Accordingly, conventional disk joints can limit linear motion of the knee brace wearer's knee which can unnecessarily load and/or strain the knee's soft tissues.

By contrast to conventional knee braces, embodiments of the present innovation relate to a knee brace apparatus. In one arrangement, the knee brace apparatus includes a first brace assembly configured to be secured to a user's leg above the knee and a second brace assembly configured to be secured to the user's leg below the knee. The knee brace apparatus also includes a hinge disposed between the first brace assembly and the second brace assembly. The hinge includes a set of strap elements arranged such that the planar surfaces of the strap elements are disposed substantially parallel to each other along an anterior-posterior orientation of the knee brace apparatus. With such an arrangement, the hinge is substantially flexible along the anterior-posterior orientation and is substantially stiff along the medial-lateral orientation. Accordingly, the hinge allows a wearer to flex the knee brace apparatus at the knee joint while protecting the wearer's knee from impact loading. Additionally, as the user flexes the knee brace apparatus, the strap elements are configured to translate or slide along a longitudinal axis relative to each other. The knee brace apparatus can therefore substantially track the physiological motion of a wearer's knee, including both rotation and linear translation, during operation to minimize binding of the hinge.

In one arrangement, a knee brace assembly comprises a first brace assembly configured to be secured to a first leg portion of a leg relative to a knee, a second brace assembly configured to be secured to a second leg portion of the leg relative to the knee, the second leg portion opposing the first leg portion and a hinge disposed between the first brace assembly and the second brace assembly. The hinge includes a plurality of strap elements, each strap element defining a planar surface, the planar surfaces of the plurality of strap elements being disposed substantially parallel to each other along an anterior-posterior orientation of the first leg portion and the second leg portion.

### BRIEF DESCRIPTION OF THE DRAWINGS

The foregoing and other objects, features and advantages will be apparent from the following description of particular embodiments of the innovation, as illustrated in the accompanying drawings in which like reference characters refer to the same parts throughout the different views. The drawings are not necessarily to scale, emphasis instead being placed upon illustrating the principles of various embodiments of the innovation.

FIG. 1 illustrates a schematic, perspective view of a knee brace assembly, according to one arrangement.

FIG. 2 illustrates a front view of the knee brace assembly of FIG. 1 mounted to a wearer's leg, according to one arrangement.

FIG. 3 illustrates a side view of the knee brace assembly of FIG. 1 mounted to a wearer's leg, according to one arrangement.

FIG. 4 illustrates an exploded schematic view of a set of strap elements of the knee brace assembly of FIG. 1, according to one arrangement.

FIG. 5A illustrates a side view of a set of strap elements of the knee brace assembly of FIG. 1 disposed in a first position.

FIG. 5B illustrates a side view of a set of strap elements of the knee brace assembly of FIG. 1 disposed in a second position.

FIG. 6A illustrates a side sectional view of a set of strap elements mounted to a hinge holder of the knee brace assembly of FIG. 1.

FIG. 6B illustrates a perspective view of a set of strap elements of the knee brace assembly of FIG. 1 having a pin engagement portion.

FIG. 7 illustrates a schematic sectional view of a hinge holder of the knee brace assembly of FIG. 1 having a torque resistant mechanism, according to one arrangement.

FIG. 8A illustrates a front view of the knee brace assembly of FIG. 1 having a torque resistant mechanism, according to one arrangement.

FIG. 8B illustrates a side view of the knee brace assembly of FIG. 8B having the torque resistant mechanism, according to one arrangement.

FIG. 9 illustrates a schematic, perspective view of a knee brace assembly, according to one arrangement.

FIG. 10 illustrates a side view of the knee brace assembly of FIG. 9, according to one arrangement.

FIG. 11A illustrates a side view of an energy absorption member.

FIG. 11B illustrates a side view of an energy absorption member.

FIG. 12 illustrates a tool used to insert the energy absorption member of FIGS. 11A and 11B into ring elements of a brace assembly.

FIG. 13 is a partial cross sectional view of a brace assembly using the energy absorption members of FIGS. 11A and 11B.

FIG. 14 illustrates a front view of the knee brace assembly mounted to a wearer's leg, according to one arrangement.

### DETAILED DESCRIPTION

Embodiments of the present innovation relate to a knee brace apparatus. In one arrangement, the knee brace apparatus includes a first brace assembly configured to be secured to a user's leg above the knee and a second brace assembly configured to be secured to the user's leg below the knee. The knee brace apparatus also includes a hinge disposed between the first brace assembly and the second brace assembly. The hinge includes a set of strap elements arranged such that the planar surfaces of the strap elements are disposed substantially parallel to each other along an anterior-posterior orientation of the knee brace apparatus. With such an arrangement, the hinge is substantially flexible along the anterior-posterior orientation and is substantially stiff along the medial-lateral orientation. Accordingly, the hinge allows a wearer to flex the knee brace apparatus at the knee joint while protecting the wearer's knee from impact loading. Additionally, as the user flexes the knee brace apparatus, the strap elements are configured to translate or slide along a longitudinal axis relative to each other. The knee brace apparatus can therefore substantially track the physiological motion of a wearer's knee, including both rotation and linear translation, during operation to minimize binding of the hinge.

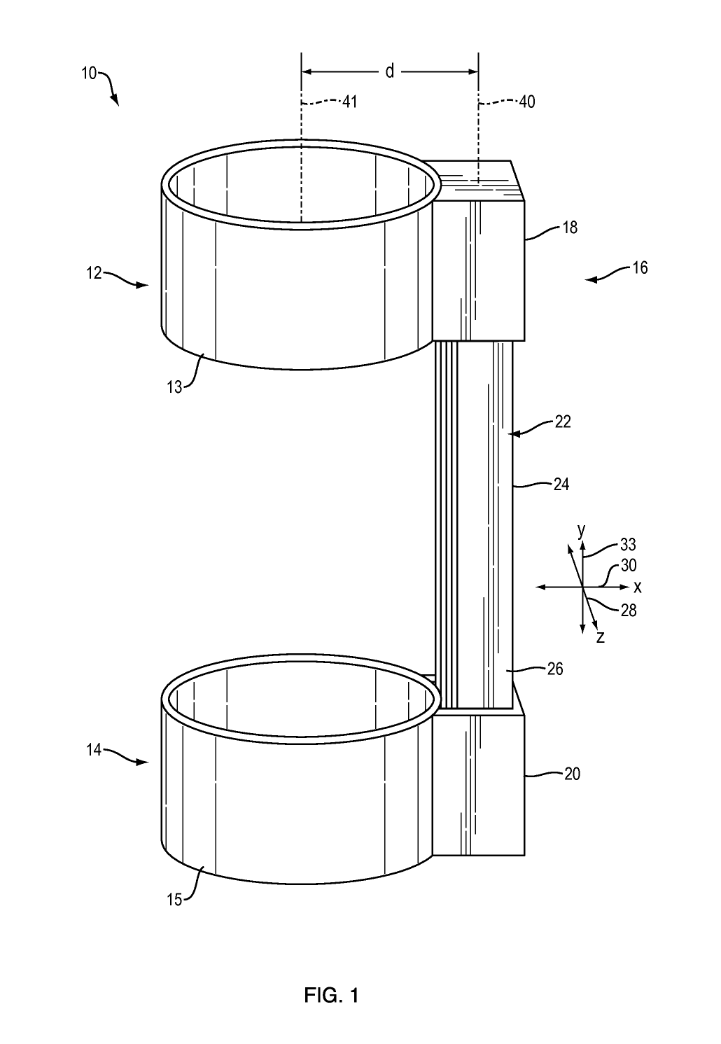

FIGS. 1-3 illustrate a knee brace apparatus 10, according to one arrangement. The knee brace apparatus 10 includes a first brace assembly 12, a second brace assembly 14, and a hinge 16 disposed between the first brace assembly 12 and the second brace assembly 14.

In one arrangement, the first brace assembly 12 is configured to be secured to a wearer's upper leg or thigh above the wearer's knee 27 and the second brace assembly 14 is configured to be secured to a wearer's lower leg below the wearer's knee 27. It should be noted that the first and second brace assemblies 12, 14 can be secured to the wearer's leg in a variety of ways. For example, one or both of the first and second brace assemblies 12, 14 can be mechanically attached to the wearer's pants above and below the wearer's knee 27. In another example, the first and second brace assemblies 12, 14 can be secured to the wearer's leg via a friction fit, either over or under the wearer's pants. While the brace assemblies 12, 14 can be manufactured from a variety of materials, in one arrangement, the assemblies 12, 14 are manufactured from either a cloth material or substantially rigid plastic material.

Each of the first brace assembly 12 and the second brace assembly 14 can include a sizing mechanism (not shown) configured to adjust the circumference of the first and second brace assemblies 12, 14 to conform to the wearer's leg 25. For example, in the case where the brace assemblies 12, 14 are manufactured from a cloth material, the sizing mechanisms can be configured as one or more hook and loop material (e.g., VELCRO) straps. In another example, in the case where the brace assemblies 12, 14 are manufactured from a plastic material, the sizing mechanisms can be configured as a compliant material, such as a foam rubber material, which extends about the inner peripheries of the housings 13, 15 of the assemblies 12, 14. In such a case, the compliant material reduces an inner diameter of the housings 13, 15 to create a friction fit between the assemblies 13, 15 and the wearer's leg 25. Alternately, the first and second assemblies 13, 15 can each include an inflatable cuff or bladders as the sizing mechanism. For example, after placing the knee brace apparatus 10 on his leg 25, the wearer can inflate each bladder of the first and second brace assemblies 12, 14 to create a friction fit between the knee brace apparatus 10 and the wearer's leg 25.

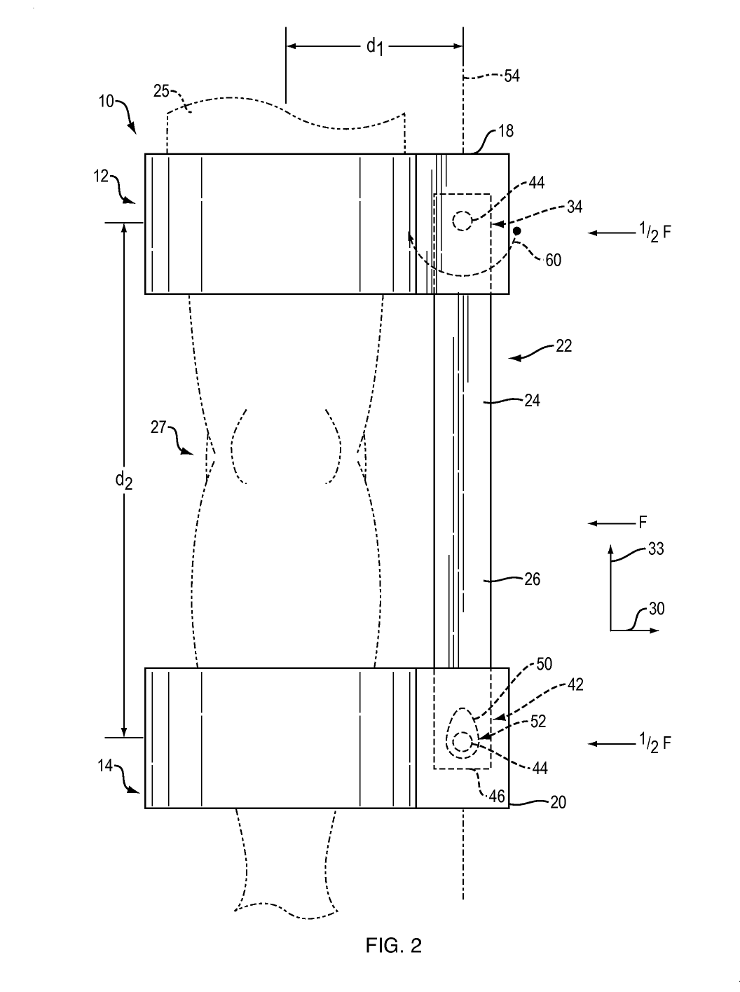

As illustrated, each of the first and second brace assemblies 12, 14 include respective first and second hinge holders 18, 20 configured to secure opposing ends of the hinge 16 to the first and second brace assemblies 12, 14. For example, with particular reference to FIG. 2, the first and second hinge holders 18, 20 are disposed at a lateral location on the knee brace apparatus 10. As illustrated, the hinge holders 18, 20 define a longitudinal axis 40 which is offset by a distance d₁ from a centerline or longitudinal axis 41 defined by the first and second brace assemblies 12, 14. With such a configuration, and with continued reference to FIG. 2, when the knee brace apparatus 10 is placed on a wearer's left leg 25, the first and second hinge holders 18, 20 maintain the hinge 16 at a lateral position on a wearer's knee (i.e., on the outside of the wearer's leg 25) to minimize interference between the hinge 16 and the wearer.

In order to maintain consistency in the placement and orientation of the knee brace apparatus 10 on the wearer's leg, the hinge 16 is configured to maintain a longitudinal spacing d₂ between the first and second brace assemblies 12, 14. For example, with first and second ends of the hinge 16 disposed within the respective first and second hinge holders 18, 20, the hinge 16 maintains a substantially fixed longitudinal spacing or distance d₂ between the first and second brace assemblies 12, 14.

The hinge 16 is further configured to provide relative motion between the first and second brace assemblies 12, 14 when the wearer flexes his leg 25 at the knee 27. In one arrangement, as shown in FIGS. 1, 3, and 4, the hinge 16 includes a set of strap elements 22, each strap element 24 of the set 22 being configured as a generally rectangular structure and defining planar surfaces such as an anterior surface 26 and a posterior surface 29. While the strap elements 24 can be manufactured from a variety of materials, in one arrangement, each of the strap elements 24 can be manufactured from a plastic material or from a metallic material.

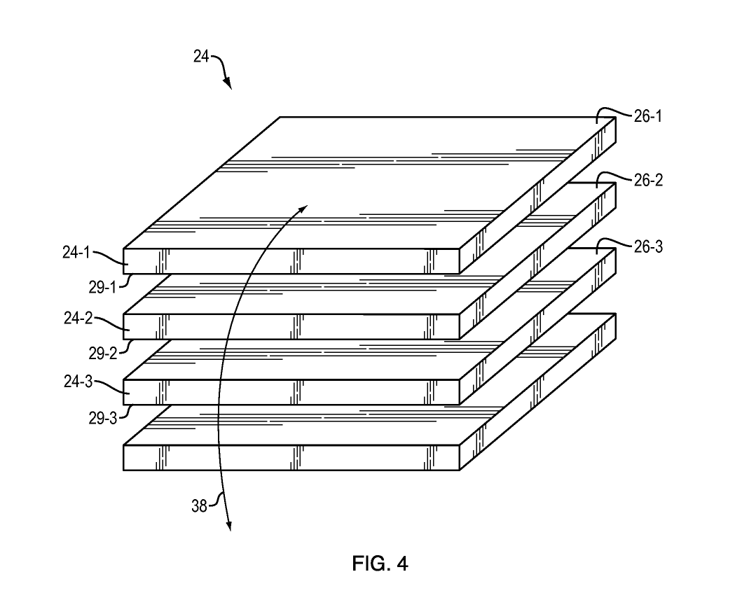

The opposing ends of the set of strap elements 22 are disposed within the respective first and second hinge holders 18, 20 such that opposing anterior and posterior surfaces 26, 29 of adjacent strap elements 24 are disposed in a stacked arrangement substantially parallel to each other along an anterior-posterior orientation 28 of the knee brace apparatus 10. For example, with reference to FIG. 4, a first strap element 24-1 defines a first anterior and posterior surface 26-1, 29-2, a second strap element 24-2 defines a second anterior and posterior surface 26-2, 29-2, and a third strap element 24-3 defines a third anterior and posterior surface 26-3, 29-3. As shown, the second anterior surface 26-2 of the second strap element 24-2 opposes the first posterior surface 29-1 of the first strap element 26-1 and the second posterior surface 29-2 of the second strap element 24-2 opposes the first anterior surface 26-3 of the third strap element 26-3. Accordingly, during operation, as the strap elements 24 bend about arc 38, the opposing anterior and posterior surfaces 26, 29 remain substantially parallel to each other.

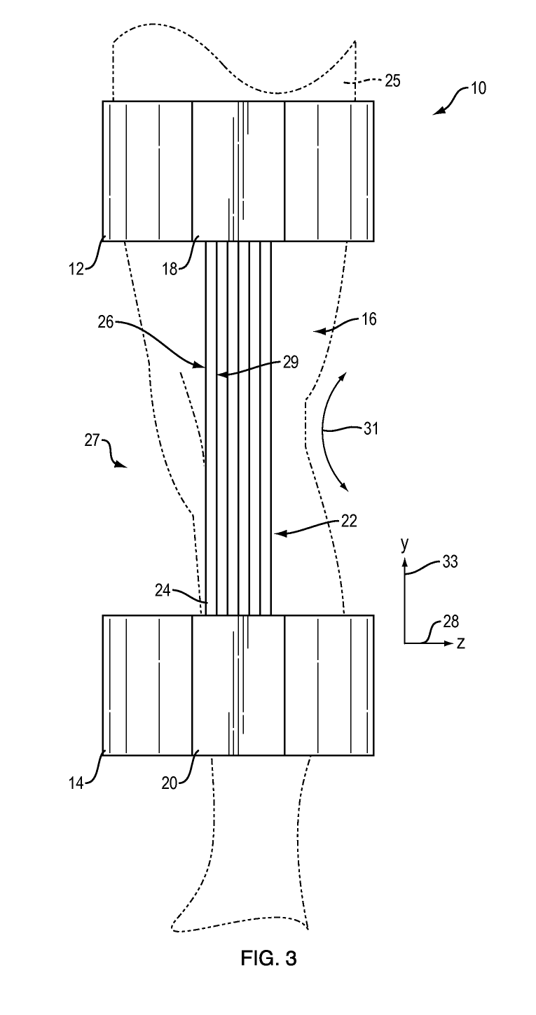

Based upon the geometric configuration of the strap elements 24 of the hinge 16, as well as the material properties and the orientation of the hinge 16 relative to the first and second brace assemblies 12, 14, the hinge 16 is substantially flexible along an anterior-posterior direction or axis 28. During operation, as the wearer flexes or extends his leg 25 (e.g., along arc 31 as shown in FIG. 3), the set of strap elements 22 can flex or bend along the anterior-posterior axis 28. Accordingly, the hinge 16 allows a wearer to flex the knee brace apparatus 10 about his knee joint while protecting the wearer's knee 27 when exposed to medial-lateral loading.

The relative flexibility of the knee brace apparatus 10 along an anterior-posterior axis or direction 28 can depend upon the material properties of the strap elements 24 of the hinge 16. For example, to configure the hinge 16 as relatively compliant or flexible along the anterior-posterior axis 28, a manufacturer can select relatively compliant plastic strap elements 24, relatively compliant metallic strap elements 24, or some combination thereof to form the hinge 16. In another example, to configure the hinge 16 as relatively stiff along the anterior-posterior axis 28, a manufacturer can select relatively stiff plastic strap elements 24, relatively stiff metallic strap elements 24, or some combination thereof to form the hinge 16.

In use, while the first and second brace assemblies 12, 14 are secured to the wearer's leg, as the wearer flexes and extends his leg, the positioning of the first and second brace assemblies 12, 14 relative to each other can vary. To account for this change in relative positioning, the hinge 16 can be physically configured to allow for relative movement of the first and second brace assemblies 12, 14 during use.

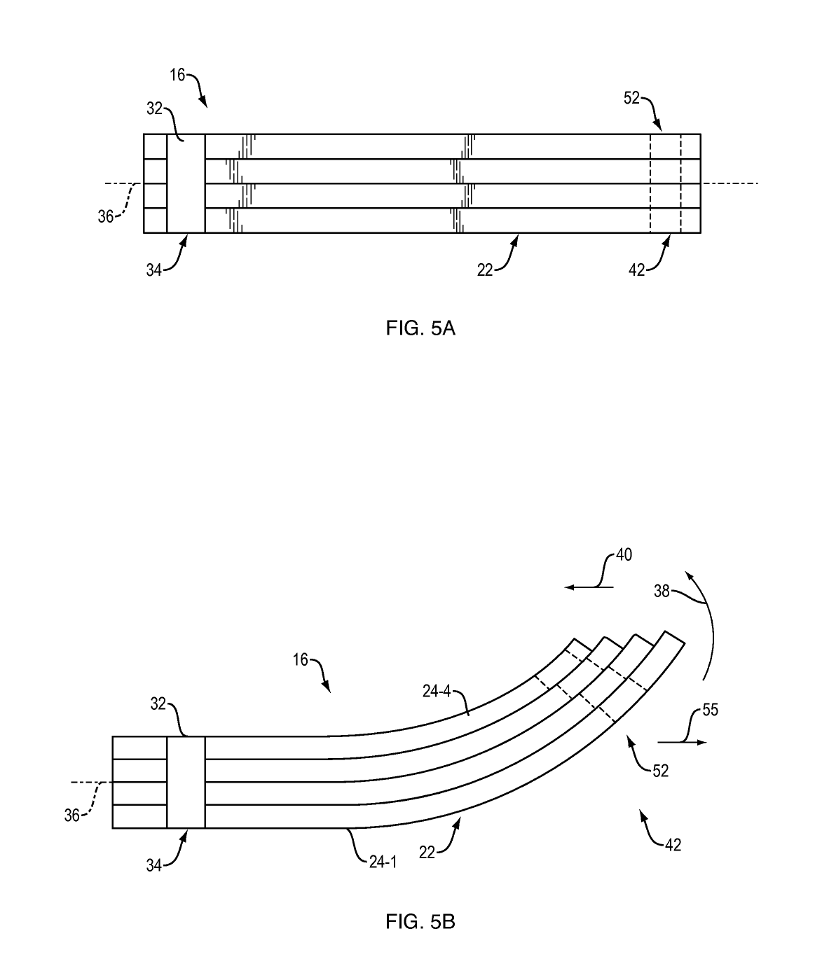

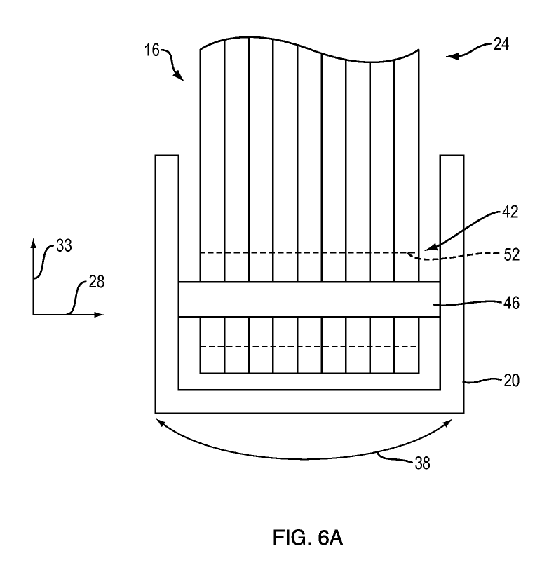

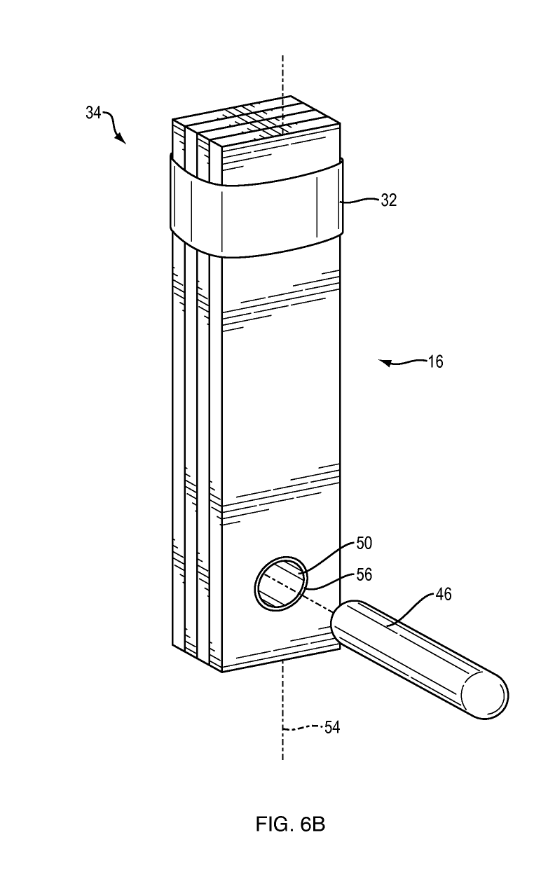

For example, with reference to FIGS. 5A, 5B, and 6B, the hinge 16 can include a binding member 32 disposed at a first end 34 of the hinge 16 which is configured to secure the plurality of strap elements 24 together at the first end 34. The binding member 32 can be configured in a variety of ways. For example, the binding member 32 can be configured as a band disposed about the plurality of strap elements 24 to secure the strap elements 24 together. In another example, the binding member 32 can be configured as an adhesive disposed at the first end 34 between adjacent strap elements 24.

By securing the strap elements 24 at the first end 34, the binding member 32 allows the plurality of strap elements 22 to translate or slide relative to each other along a longitudinal axis 36, as illustrated in FIGS. 5A and 5B. For example, in use, as the hinge 16 rotates along arc 38, the posterior strap elements 24-4 can translate (e.g., shrink) along direction 40 at a second end 42 of the hinge 16 while the anterior strap elements 24-1 can translate (e.g., stretch) along direction 55. This relative translation of the strap elements 24 allows a change in the relative positioning of the first and second brace assemblies 12, 14 and minimizes binding of the hinge 16 during operation.

With such a configuration, and with reference to FIG. 2, the hinge 16 can be mounted within the knee brace apparatus 10 such that the first end 34 is fixedly coupled to the first brace assembly 12 and the second end 42 is moveably coupled to the second brace assembly 14. For example, the first end 34 of the hinge 16 can be affixed to the first hinge holder 18 via a first pin assembly 44 and the second end 42 of the hinge 16 can be moveably mounted to the second hinge holder 20 via a second pin assembly 46 and a translation mechanism 50.

In one arrangement, with reference to FIGS. 2 5A, 5B, and 6A, the translation mechanism 50 is configured as an opening 52 defined by the strap elements 24 and elongated along a longitudinal axis 54 of the hinge 16. As indicated in FIG. 6A, the second pin assembly 46 is connected to the second hinge holder 20. When the second pin assembly 46 is disposed within the opening 52, the assembly 46 secures the second end 42 of the hinge 16 to the second hinge holder 20. During operation, and with reference to FIGS. 5A, 5B, and 6A, when the hinge 16 flexes along arc 38 and along an anterior-posterior orientation 28 of the knee brace apparatus 10, the strap elements 24 translate relative to each other and translate relative to a longitudinal axis of the pin assembly 46. Accordingly, interaction of the pin assembly 46 and the elongated opening 52 secures the second end 42 of the hinge 16 to the second hinge holder 20 while allowing movement and minimizing binding of the strap elements 24.

In another arrangement, with reference to FIG. 6B, the translation mechanism 50 is configured as an elastomeric member 56. As indicated, when the second pin assembly 46 is disposed within the opening 52, the second pin assembly 46 is also at least partially surrounded by the elastomeric member 56 to secure the second end 42 of the hinge 16 to the second hinge holder 20. Because the elastomeric member 56 surrounds the second pin assembly 46, as the strap elements 24 translate relative to each other during operation, the second pin assembly 46 can compress and elongate the elastomeric member 56 along the longitudinal axis 54 to minimize binding of the strap elements 24.

As indicated above, the hinge 16 is configured as being substantially flexible along the anterior-posterior direction or axis 28. Further, with reference to FIGS. 1 and 4, because the strap elements 24 are disposed in a stacked arrangement, the hinge 16 is configured with a relatively high stiffness along a medial-lateral axis 30. Accordingly, when exposed to a loading directed substantially along the medial lateral axis 30, the hinge 16 is configured to distribute the load through the knee brace apparatus 10.

For example, with reference to FIG. 2, in the case where the hinge 16 receives a load, such as an impact load, directed substantially along the medial-lateral axis 30, the hinge 16 is configured to transmit and distribute the load, as about ½ F, to each of the first and second brace assemblies 12, 14. Accordingly, by distributing the load F to the first and second brace assemblies 12, 14, the hinge 16 causes the wearer's upper and lower leg portions to receive and distribute a reduced impact load and minimizes injury to the wearer's knee as caused by an impact.

As described above, the hinge 16 is substantially flexible along an anterior-posterior orientation and is substantially rigid along a medial-lateral orientation. Accordingly, the hinge 16 allows a wearer to flex the knee brace apparatus 10 at his knee joint 27 while substantially tracking the physiological motion of a wearer's knee, including both rotation and linear translation of the upper and lower leg portions relative to each other. Therefore, the hinge 16 minimizes binding of the knee brace apparatus 10 limits the apparatus 10 from generating load and/or strain on the soft tissues of the wearer's knee. Further, the hinge 16 is configured to protect the wearer's knee 27 from medial-lateral impact loading. With the orientation and stacked arrangement of the strap elements 24 within the knee brace apparatus, the hinge 16 is configured with a relatively high stiffness along a medial-lateral axis 30. Such a configuration reduces a medial-lateral impact load received by the wearer and minimizes injury to the wearer's knee as caused by the impact.

As indicated above, the knee brace apparatus 10 is configured to allow a wearer to flex his knee at the knee joint 27 while protecting the wearer's knee from impact loading. During use, the wearer's knee may be exposed to a torque load. In one arrangement, the knee brace apparatus 10 can include a torque reducing mechanism configured to limit or minimize torqueing of the wearer's upper and lower leg portions about the knee joint.

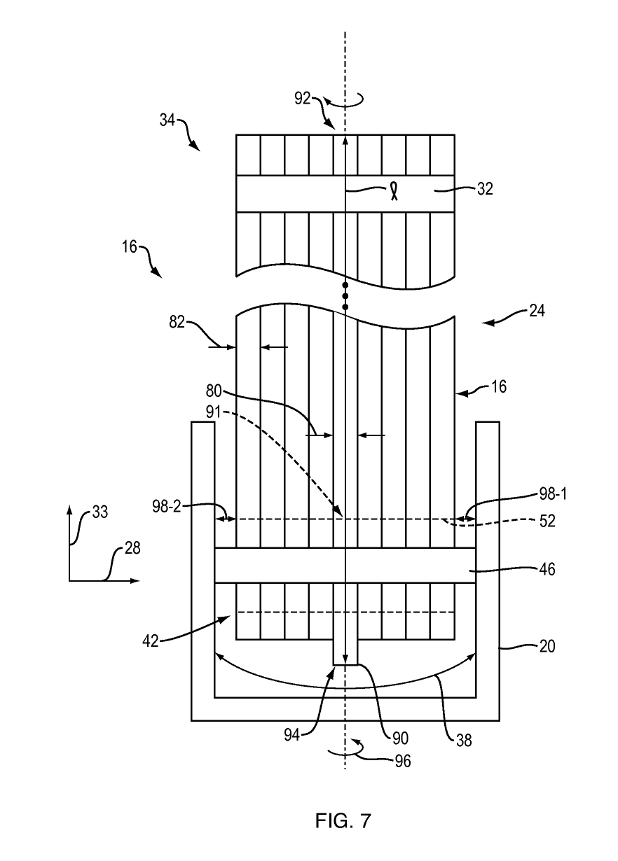

FIG. 7 illustrates a schematic, sectional view of a torque reducing mechanism 90, according to one arrangement. As illustrated, the torque reducing mechanism 90 is configured as a generally rectangular structure which forms part of the hinge 16. For example, the torque reducing mechanism 90 can be disposed at a substantially central location relative to the strap elements 24 of the hinge 16. The torque reducing mechanism 90 includes a first end 92 secured to the first end 34 of the hinge 16 with binding member 32 and a second end 94 which defines an elongated opening 91 which is substantially aligned with the elongated opening 52 defined by the strap elements 24. The second pin assembly 46 is disposed within the opening 52 defined by the strap elements 24 and within the opening 91 defined by the torque reducing mechanism 90 to secure the second end 42 of the hinge 16 to the second hinge holder 20. With such a configuration, interaction of the pin assembly 46 and the elongated openings 52, 91 secures the second end 42 of the hinge 16 to the second hinge holder 20 while allowing the strap elements 24 and the torque reducing mechanism 90 to translate within the second hinge holder 20 along distances 98-1, 98-2 during operation while minimizing binding.

To minimize the distribution of a torque load 96 to a wearer's knee, the torque reduction mechanism 90 is configured with a relatively larger torsional rigidity or stiffness than the strap elements 24. For example, in the case where the strap elements 24 are manufactured from a first material, such as a plastic material, having a first stiffness, the torque reduction mechanism 90 can be manufactured from a second material, such as a metal material having a second, higher stiffness. In another example, assume the case where the strap elements 24 and the torque reduction mechanism 90 are manufactured from the same material, such as a plastic material. In such a case, the thickness 80 of the torque reduction mechanism 90 can be greater than the thickness 82 of the strap elements 24. The relative difference in thickness provides the torque reduction mechanism 90 with a relatively larger torsional rigidity compared to the strap elements 24. In another example, the torque reducing mechanism 90 defines a length l that is longer than the lengths of the strap elements 24 of the hinge 16. The difference in length provides the torque reduction mechanism 90 with a relatively larger torsional rigidity compared to the strap elements 24.

In any of the example configurations provided, the torque reducing mechanism 90 is configured to flex or bend with the strap elements 24 along arc 38 and substantially within an anterior-posterior orientation 28 of the knee brace apparatus 10. However, because the torque reduction mechanism 90 is configured with a relatively larger torsional rigidity compared to the strap elements 24, the torque reduction mechanism 90 can resist torsional bending in response to a load 96 applied to opposing ends 34, 42 of the hinge 16. Accordingly, the torque reduction mechanism 90 can aid the knee brace apparatus 10 in minimizing the risk of injury to a wearer's knee when exposed to a torque load.

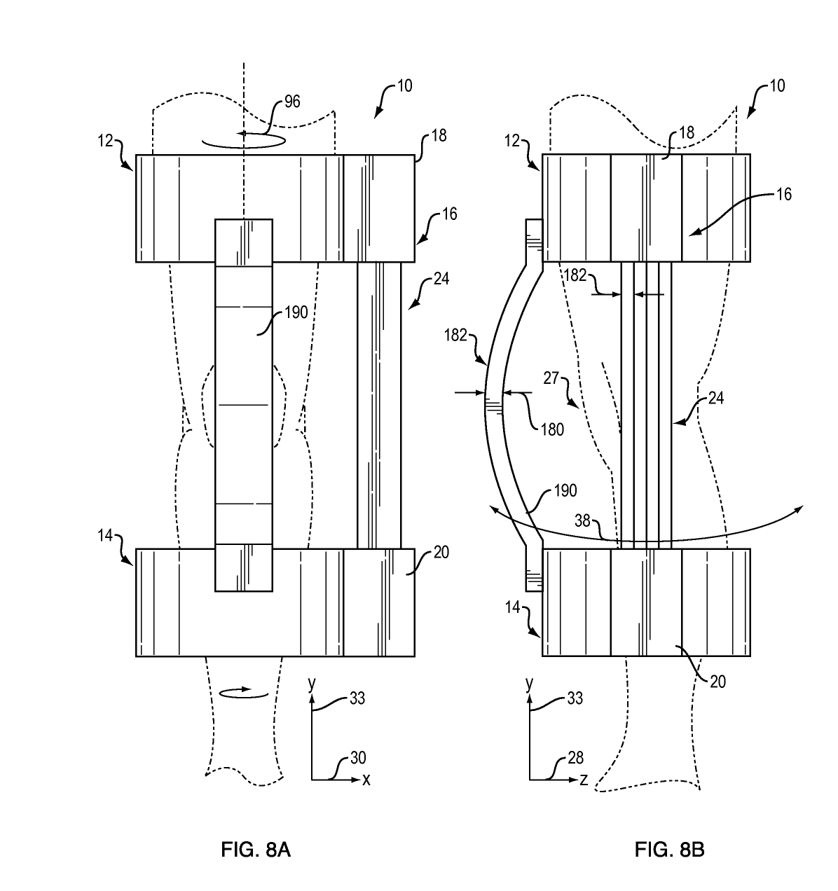

FIGS. 8A and 8B illustrate a front and side view, respectively, of a torque reducing mechanism 190 associated with the knee brace apparatus 10. As illustrated, the torque reducing mechanism 190 is configured as a generally rectangular structure connected between the first and second brace assemblies 12, 14. While FIGS. 8A and 8B illustrate the torque reducing mechanism 190 as connected to the first and second brace assemblies 12, 14, it should be understood that the torque reducing mechanism 190 can also be connected between the first and second hinge holders 18, 20.

As shown in FIG. 8B, the torque reducing mechanism 190 can define a curved portion 182 which is spaced at a radial distance from a wearer's knee 27. During operation, the radial spacing of the torque reducing mechanism 190 minimizes contact between the knee brace apparatus 10 and the wearer's knee 27 as the wearer flexes or extends his leg along arc 38 in the Y-Z plane.

To minimize the distribution of a torque load 96 to a wearer's knee, the torque reduction mechanism 190 is configured with a relatively larger torsional rigidity or stiffness than the hinge 16. For example, in the case where the strap elements 24 of the hinge 16 are manufactured from a first material, such as a plastic material, having a first stiffness, the torque reduction mechanism 190 can be manufactured from a second material, such as a metal material having a second, higher stiffness. In another example, assume the case where the strap elements 24 of the hinge 16 and the torque reduction mechanism 90 are manufactured from the same material, such as a plastic material. In such a case, the thickness 180 of the torque reduction mechanism 90 can be greater than the thickness 182 of the strap elements 24. The relative difference in thickness provides the torque reduction mechanism 190 with a relatively larger torsional rigidity compared to that of the set strap elements 22 of the hinge 16.

In either configuration, the torque reducing mechanism 190 is configured to flex or bend with the hinge 16 along arc 38 and substantially within an anterior-posterior orientation 28 of the knee brace apparatus 10. However, because the torque reduction mechanism 190 is configured with a relatively larger torsional rigidity compared to the hinge 16, the torque reduction mechanism 190 can resist torsional bending in response to a load 96 applied to opposing ends 34, 42 of the hinge 16. Accordingly, the torque reduction mechanism 190 can aid the knee brace apparatus 10 in minimizing the risk of injury to a wearer's knee when exposed to a torque load.

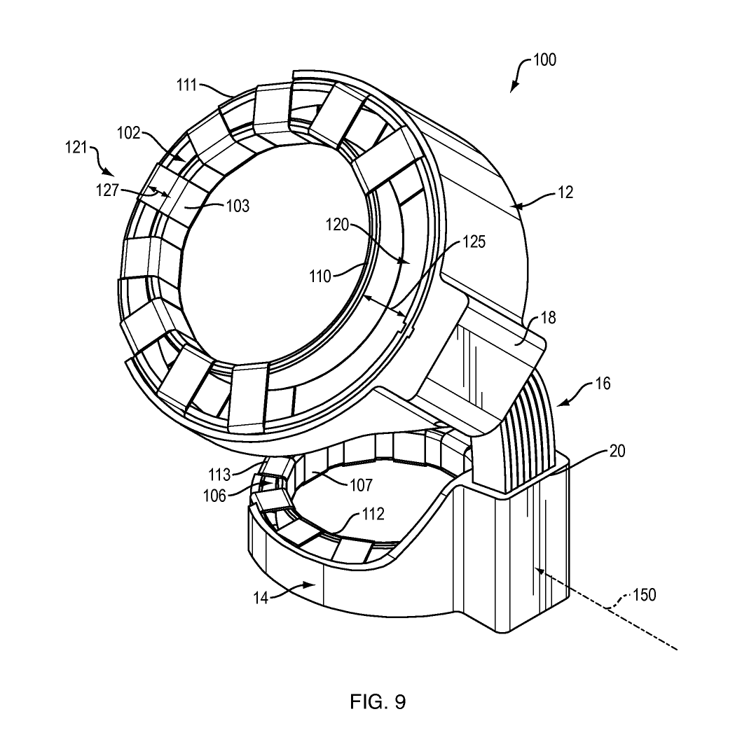

As indicated above, the first and second brace assemblies 12, 14 can include a sizing mechanism configured to adjust the circumference of the first and second brace assemblies 12, 14 to conform to the wearer's leg 25. In one arrangement, as illustrated in FIGS. 9 and 10, a device 100 is configured to absorb the energy of impacts to the wearer's leg, outside of the wearer's knee. As illustrated, the first and second brace assemblies 12, 14 can include energy absorption subassemblies configured to absorb at least a portion the energy of impact loads directed to areas of a wearer's leg proximal or distal to the wearer's knee.

FIGS. 9 and 10 illustrate a knee brace assembly 100 having first and second brace assemblies 12, 14 interconnected by a hinge 16 which allows assemblies 12, 14 to pivot about one or more axes that are substantially normal to the surface of hinge 16, as described above. The first and second brace assemblies 12, 14 each include respective energy absorption subassemblies 102, 106.

For example, each of the energy absorption subassemblies 102, 106 can include respective first or inner ring elements 110, 112 and second or outer ring elements 111, 113. The energy absorption subassemblies 102 and 106 also include respective first and second energy-absorption members 103, 107. As illustrated, with respect to the first brace assembly 12, the first energy absorption member 103 span the distance between and connects the first ring element 110 to the second ring element 111 and, with respect to the second brace assembly 14, the second energy absorption member 107 spans the distance between and connects the first ring element 112 to the second ring element 113. The elastomeric energy-absorption members 103, 107 are configured to expand and contract between the respective first 110, 112 and second 111, 113 ring elements in response to loading of the first and second brace assemblies 12, 14.

While the energy absorption members 103, 107 can be configured in a variety of ways, in one arrangement, the energy-absorption members 103, 107 are constructed as annular pieces of elastomeric material. The energy absorption members 103, 107 can be created, for example, through extrusion or by cutting an elastomeric tube of the correct diameter into pieces of a desired width.

The energy absorption members 103, 107 can be disposed at particular locations, and spaced in a particular manner, to accomplish a given amount of energy-absorption at one or more locations on the energy absorption subassemblies 102, 106. For example, stronger elastomers can be placed with some slack such that they begin to stretch only close to the endpoint of travel of the outer rings 111, 113. In another example, multiple elastomeric energy absorption members 103, 107 of different lengths and/or different strengths can be located in parallel so that their energy-absorption is cumulative.

Assemblies 102 and 106 are arranged such that in the rest position shown in the drawings, there is a larger gap between the respective inner 110, 112 and outer rings 111, 113 at a lateral location 120 than at a medial location 121. Taking the first brace assembly as an example, a first gap 125 between the first and second ring elements 110, 111 at lateral location 120 is larger than a second gap 127 between the first and second ring elements 110, 111 at the opposing, medial location 121. Since the gap 125 in the area of impact defines the maximum travel of the outer rings 111, 113 of the energy-absorption subassemblies 102, 106 relative to the inner rings 110, 112, having the inner 1110, 112 and outer rings 111, 113 generally but not exactly concentric, as in this case, can provide additional energy absorption in one direction, which in this case is impact to the outside of the knee area that can cause severe injury.

In use, and with particular reference to FIG. 10, the hinge 16 is placed proximate a knee area 132 of leg 130. The first brace assembly 12 is located above the knee, in thigh area 134. The second brace assembly 14 is located below the knee, in calf area 136. The device 100 is worn such that the hinge 16 is located along the outside as opposed to the inside of the wearer's knee, where impact is most likely to occur in a sport such as football. The first and second brace assemblies 12, 14 help to transfer force at any location along the length of the assembly to one or both of the energy-absorption subassemblies 102 and 106.

For example, in the case where the second brace assembly 14 receives an impact load along direction 150, as shown in FIG. 9, the load causes the energy-absorption members 103, 107 disposed along the medial location 121 of the device 100 to expand between the first ring element 113 and the second ring element 112 in response to a relative translation of the first ring element 113 and the second ring element 112. Such expansion of the energy-absorption members 107 causes the device to absorb the impact load and protect the wearer's knee 132.

While various embodiments of the innovation have been particularly shown and described, it will be understood by those skilled in the art that various changes in form and details may be made therein without departing from the spirit and scope of the innovation as defined by the appended claims.

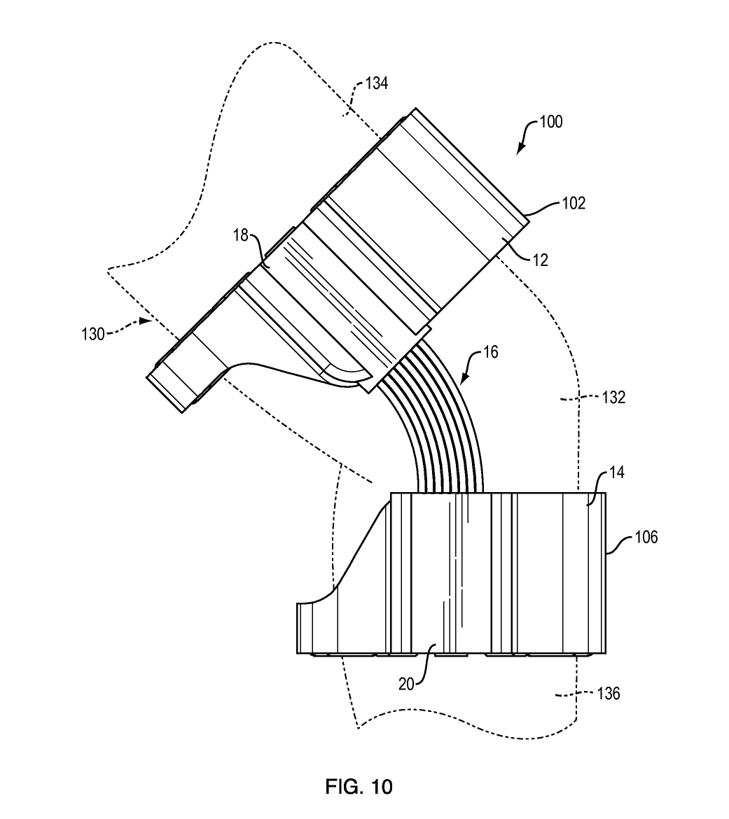

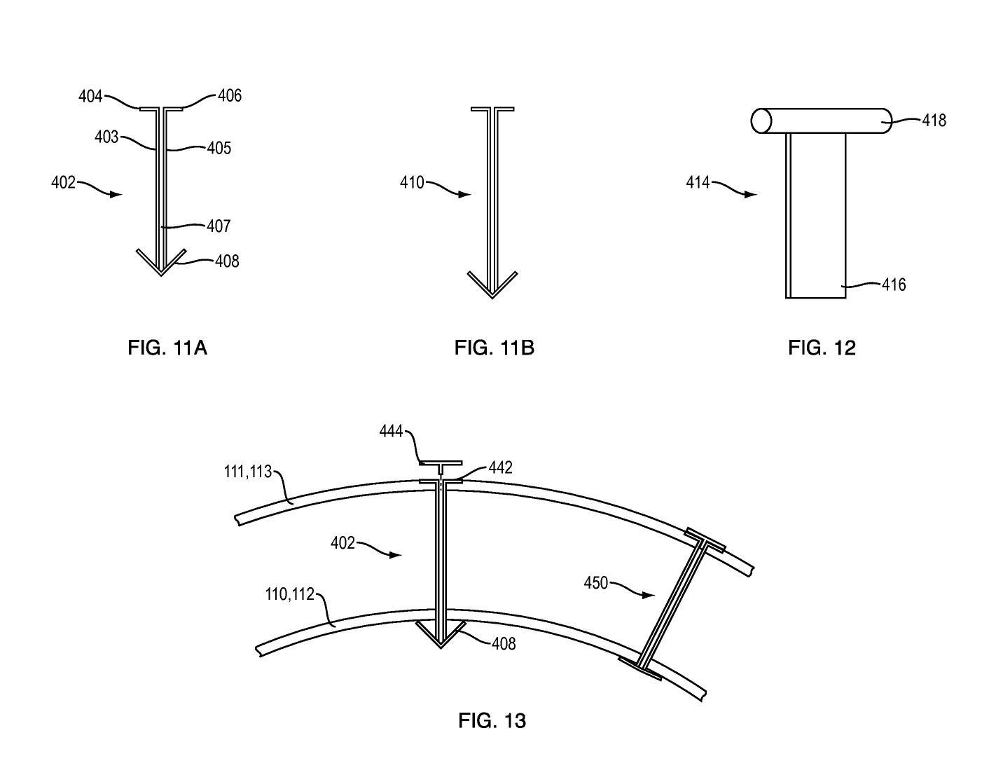

For example, as described above, the energy absorption members 103, 107 are described as annular pieces of elastomeric material. Such description is by way of example only. In one arrangement, the energy absorption members can be configured as insertable elastomers, as illustrated in FIGS. 11A through 13.

FIGS. 10A and 10B show two energy absorption members 402 and 410. In one arrangement, the differences between the energy absorption members 402, 410 can include the length and/or strength of the members. The energy absorption members 402, 410 include parallel legs 403 and 405 that have substantially perpendicular terminal portions 404 and 406 and distal terminal portion 408. The energy absorption members 402 and 410 can be coupled to two spaced ring elements such as ring elements 111, 110 or ring elements 113, 112 of the knee brace assembly 10.

During assembly, the energy absorption members 402, 410 are pushed through aligned openings in the ring elements via tool 414, illustrated in FIG. 12. In one arrangement, the tool 414 includes a blade 416 that is sized and shaped to fit into opening 407 between legs 403 and 405. During assembly, the handle 418 is pushed down to force enlarged end 408 through holes in the outer ring elements 113, 112 and corresponding inner ring elements 111, 110. Upper ends 404 and 406 sit against the outer ring elements 113, 112 adjacent to the opening. This anchors the members 402, 410 to both ring elements. As shown in FIG. 13, enlarged common end 408 of member 402 sits against the inside of inner ring element 110 while end 442 sits in a recess on the outside of outer ring element 111. A cap 444 can be pushed into the recess to further secure the energy absorption member 402 to the outer ring element 111. In one arrangement, energy absorption member 450 is slightly longer than member 402 so it is slack in the at-rest, non-impacted position depicted in FIG. 13.

Upon impact, member 402 will be stretched and then eventually, if the inner and outer ring elements are moved sufficiently far apart, member 450 can be stretched to absorb more energy. Also, as described above, the different members can be different strengths (e.g., different thicknesses) to provide more variability to the energy absorption characteristics of the protective device.

As indicated above, the hinge 16 can be mounted within the knee brace apparatus 10 such that the first end 34 is fixedly coupled to the first brace assembly 12. In one arrangement, with reference to FIG. 2, the first end 34 of the hinge 16 is coupled to the first brace assembly 12 by a pin assembly 44 that allows the hinge 16 to rotate along an arc 60 substantially along a medial-lateral axis 30 relative to the first brace assembly. Such a configuration allows relative movement of the wearer's lower and upper leg portions during use which minimizes unnecessarily loading and/or straining of the wearer's soft tissues associated with the knee 27.

FIG. 2 illustrates the hinge 16 distributing axial loading F along axis 30. Such illustration is by way of example only. It should be understood that the hinge 16 is configured to distribute loading forces F that are directed toward the hinge 16 at an angle relative to axis 30. Based upon the angle of impact, the hinge 16 can distribute the load F in a non-uniform manner to the first and second brace assemblies 12, 14, such as ¾ F to the first brace assembly 12 and ¼ F to the second brace assembly.

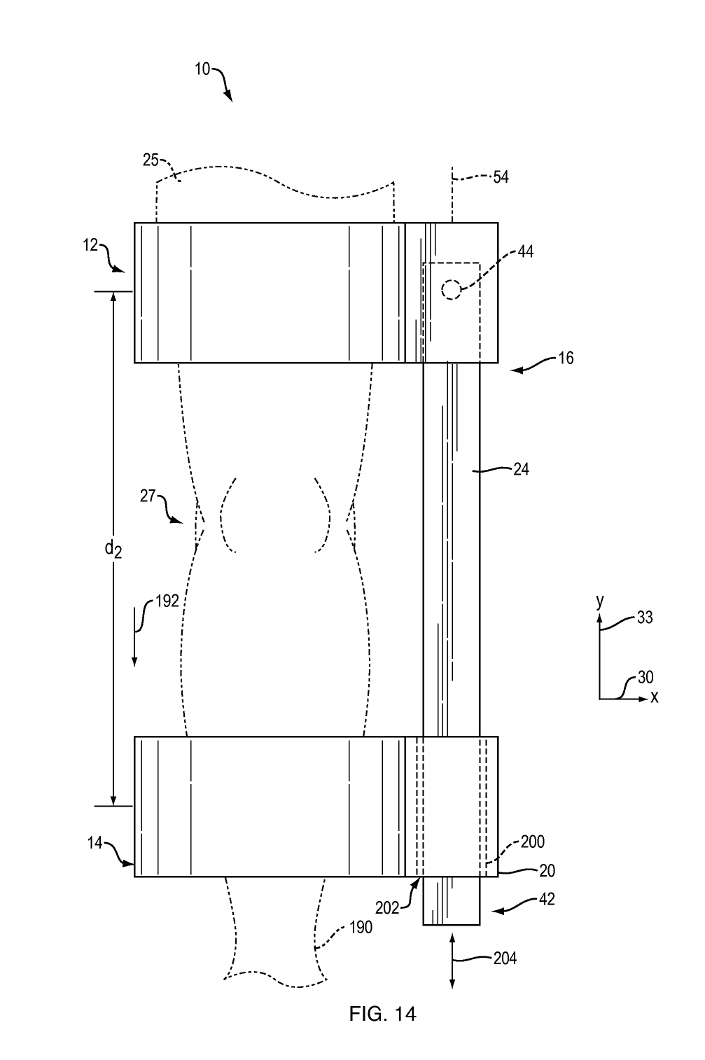

As indicated above, in order to maintain consistency in the placement and orientation of the knee brace apparatus 10 on the wearer's leg, the hinge 16 is configured to maintain a longitudinal spacing d₂ between the first and second brace assemblies 12, 14. For example, the first end 34 of the hinge 16 is connected to the first hinge holder 18 and the second end 42 of the hinge 16, is connected to the second hinge holder 20 via a second pin assembly 46. The configuration of the knee brace apparatus 10 maintains a substantially fixed longitudinal spacing or distance d₂ between the first and second brace assemblies 12, 14. Such description is by way of example only. In one arrangement, as illustrated in FIG. 14, the mounting location of the knee brace apparatus 10 maintains the longitudinal spacing d₂ between the first and second brace assemblies 12, 14.

As illustrated, the first brace assembly 12 is secured to a wearer's upper leg or thigh above the wearer's knee 27 and the second brace assembly 14 is disposed below the wearer's knee 27 and is secured above the wearer's calf 190. With the positioning of the second brace assembly 14 below the knee 27 and above the calf 190, the calf 190 limits the second brace assembly 14 from sliding along direction 192. Accordingly, the positioning of the second brace assembly 14 above the calf 190 substantially maintains the longitudinal spacing d₂ between the first and second brace assemblies 12, 14.

Additionally, because the interaction between the second brace assembly 14 and the wearer's calf 190 substantially maintains the relative positioning of the first and second brace assemblies 12, 14, the second end 42 of the hinge 16, with or without the torque reducing mechanism 90 of FIG. 7, can remain unconstrained within the second hinge holder 20. For example, as shown in FIG. 14, the second hinge holder 20 defines an opening 200 that extends there through along longitudinal axis 54. With this arrangement, the second end 42 of the hinge 16 can extend through the opening 200 and past a distal face 202 of the second hinge holder 20.

In use, as the wearer flexes or extend his leg, the strap elements 24 can translate along axis 204 and can move longitudinally (i.e., along a direction that is substantially into and out of the page) within the second hinge holder 20. Accordingly, by providing lateral and longitudinal freedom of movement of the second end 42 of the hinge 16, the opening 200 minimizes binding of the strap members 24 during operation.

What is claimed is:

1. A knee brace assembly, comprising:
a first brace assembly configured to be secured to a first leg portion of a leg relative to a knee;
a second brace assembly configured to be secured to a second leg portion of the leg relative to the knee, the second leg portion opposing the first leg portion; and
a hinge disposed between the first brace assembly and the second brace assembly, the hinge having a plurality of strap elements, each strap element defining a planar surface, the planar surfaces of the plurality of strap elements being disposed substantially parallel to each other along an anterior-posterior orientation of the first leg portion and the second leg portion.

2. The knee brace assembly of claim 1, wherein the plurality of strap elements are configured as having a first stiffness along the anterior-posterior orientation of the first leg and the second leg and a second stiffness along a medial-lateral orientation of the first leg and the second leg, the first stiffness being less than the second stiffness.

3. The knee brace assembly of claim 1, comprising a binding member disposed at a first end of the hinge, the binding member configured to secure the plurality of strap elements at the first end of the hinge and to allow the plurality of strap elements at a second end of the hinge to translate along a longitudinal axis relative to each other as the first brace assembly and the second brace assembly move between a first rotational position and a second rotational position.

4. The knee brace assembly of claim 1, wherein a first end of the hinge is coupled to the first brace assembly.

5. The knee brace assembly of claim 4, wherein the first end of the hinge is coupled to the first brace assembly by a pin assembly, the hinge configured to rotate along a medial-lateral orientation about the pin assembly relative to the first brace assembly.

6. The knee brace assembly of claim 4, wherein a second end of the hinge is moveably coupled to the second brace assembly, the second end of the hinge opposing the first end of the hinge.

7. The knee brace assembly of claim 6, wherein the second brace assembly comprises a pin assembly and the second end of the hinge defines an opening elongated along a longitudinal axis of the hinge, the pin assembly at least partially disposed within the opening.

8. The knee brace assembly of claim 6, wherein the second brace assembly comprises a pin assembly and the second end of the hinge comprises an elastomeric member, the pin assembly at least partially surrounded by the elastomeric member.

9. The knee brace assembly of claim 1, wherein the hinge is disposed on a lateral portion of the first brace assembly and on a lateral portion of the second brace assembly.

10. The knee brace assembly of claim 1, wherein at least one of the first brace assembly and the second brace assembly comprises;
a first ring element,
a second ring element spaced from the first ring element; and
one or more elastomeric energy-absorption members mechanically coupled to and spanning the distance between both the first ring element and the second ring element;
at least one of the one or more elastomeric energy-absorption members configured to expand between the first ring element and the second ring element in response to a relative translation of the first ring element and the second ring element.

11. The knee brace assembly of claim 1, comprising a torque reducing mechanism connected between the first brace assembly and the second brace assembly.

12. A knee brace assembly, comprising:
a first brace assembly configured to be secured to a first leg portion of a leg relative to a knee, the first brace assembly comprising:
a first ring element,
a second ring element spaced from the first ring element, and
one or more elastomeric energy-absorption members mechanically coupled to and spanning the distance between both the first ring element and the second ring element,
at least one of the one or more elastomeric energy-absorption members configured to expand between the first ring element and the second ring element in response to a relative translation of the first ring element and the second ring element;
a second brace assembly configured to be secured to a second leg portion of the leg relative to the knee, the second leg portion opposing the first leg portion the second brace assembly comprising:
a first ring element,
a second ring element spaced from the first ring element, and
one or more elastomeric energy-absorption members mechanically coupled to and spanning the distance between both the first ring element and the second ring element,
at least one of the one or more elastomeric energy-absorption members configured to expand between the first ring element and the second ring element in response to a relative translation of the first ring element and the second ring element; and
a hinge disposed between the first brace assembly and the second brace assembly, the hinge having a plurality of strap elements each strap element defining a, the planar surfaces of the plurality of strap elements being disposed substantially parallel to each other along an anterior-posterior orientation of the first leg portion and the second leg portion.

13. The knee brace assembly of claim 12, wherein the plurality of strap elements are configured as having a first stiffness along the anterior-posterior orientation of the first leg and the second leg and a second stiffness along a medial-lateral orientation of the first leg and the second leg, the first stiffness being less than the second stiffness.

14. The knee brace assembly of claim 12, comprising a binding member disposed at a first end of the hinge, the binding member configured to secure the plurality of strap elements at the first end of the hinge and to allow the plurality of strap elements at a second end of the hinge to translate along a longitudinal axis relative to each other as the first brace assembly and the second brace assembly move between a first rotational position and a second rotational position.

15. The knee brace assembly of claim 12, wherein a first end of the hinge is coupled to the first brace assembly.

16. The knee brace assembly of claim 15, wherein the first end of the hinge is coupled to the first brace assembly by a pin assembly, the hinge configured to rotate along a medial-lateral orientation about the pin assembly relative to the first brace assembly.

17. The knee brace assembly of claim 15, wherein a second end of the hinge is moveably coupled to the second brace assembly, the second end of the hinge opposing the first end of the hinge.

18. The knee brace assembly of claim 17, wherein the second brace assembly comprises a pin assembly and the second end of the hinge defines an opening elongated along a longitudinal axis of the hinge, the pin assembly at least partially disposed within the opening.

19. The knee brace assembly of claim 17, wherein the second brace assembly comprises a pin assembly and the second end of the hinge comprises an elastomeric member, the pin assembly at least partially surrounded by the elastomeric member.

20. The knee brace assembly of claim 1, wherein the hinge is disposed on a lateral portion of the first brace assembly and on a lateral portion of the second brace assembly.
